(19) United States
(12) Patent Application Publication
(10) Pub. No.: US 2005/0085837 A1
(43) Pub. Date: Apr. 21, 2005

(54) Devices and methods for harvesting tissue

(76) Inventor: Tewodros Gedebou, Los Angeles, CA (US)

Correspondence Address: STETINA BRUNDA GARRED & BRUCKER, 75 ENTERPRISE, SUITE 250, ALISO VIEJO, CA, 92656, US

(21) Appl. No.: 10/689,003

(22) Filed: Oct. 20, 2003

(51) Int. Cl. A61B 17/32

(52) U.S. Cl. 606/167

(57) ABSTRACT

Devices and methods for harvesting segments of tissue from a patient are disclosed. According to a preferred embodiment, the device comprises elongate upper and lower housing portions defining proximal and distal ends, and a recess therebetween, the latter being operative to capture a segment of tissue therewithin. Formed on the distal most end of the device is a cutting element, which may preferably be formed form upper and lower cutting blades operative to slice or shear a layer of tissue from a tissue mass as the same are advanced therethrough. The device may be configured to harvest tissue of any of a variety of sizes and thicknesses, and may be provided with an enhanced cutting instrument to selectively control the ability of the device to slice or shear off a segment of tissue having a desired length or width.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND OF THE INVENTION

The use of tissue harvested from a patient or donor for subsequent surgical implantation is well-known in the art. In this regard, due to the substantially high degree of biocompatibility associated with the use of natural tissues, it is frequently desired to employ such tissues in a variety of surgical procedures, whether it be implants, slings, grafts and the like. Synthetic materials, in contrast, are known to have poor biocompatibility and can and frequently do create adverse side effects, such as allergic reactions or other types of undesirable immune responses.

The use of harvested tissue is particularly optimal to the extent the same can be derived directly from the host in need of such tissue. In this respect, numerous surgical procedures are deployed which utilize a patient's own tissue harvested from one part of the body and utilized at another. Such usage of tissue virtually eliminates all risks associated with synthetic materials or tissues derived from a donor, and typically can be harvested and thereafter utilized in a single surgical procedure, which thus eliminates the need to preserve or otherwise delay usage of the freshly harvested tissue. This is particularly true of surgical procedures incorporating the use of harvested tissue as slings or grafts as are extensively utilized in a variety of urologic, orthopedic, gynecologic, and cosmetic surgical procedures.

Notwithstanding the ideal nature of harvested tissue for use in such procedures, the methodology by which such tissues are extracted is problematic and suffers from numerous drawbacks. In this regard, there is no uniform manner by which a particular segment of tissue can be derived from a given patient, especially with respect to segments of tissue having a particular thickness, length and/or width. As such, all types of tissue currently harvested must be derived via a separate and distinct surgical procedure, which thus requires substantial time and effort.

Along these lines it is recognized that a high degree of precision must be exercised by the surgeon to precisely slice or shear off the amount of tissue, and more specifically fascia, needed for a given procedure, and that failure to harvest a segment of fascia that has the necessary dimensions, such as thickness, length, width, and the like, will cause such improperly harvested tissue to fail in the underlying procedure incorporating such tissue or otherwise render the tissue worthless for any purpose. Moreover, even if such method of harvesting tissue is successful, virtually all known procedures typically result in the formation of a scar, which can be highly visible and highly undesirable when fascia is harvested via long longitudinal incisions formed upon the patient's thigh, which is perhaps the most common site utilized to harvest such fascia.

As such, there is a substantial need in the art for a device and method that can quickly, accurately and uniformly harvest a segment of tissue from a patient/donor such that the same possesses exact or near-exact dimensions, and in particular possess a known thickness, length and width. There is likewise a need for such a device and method that are of exceptionally simple construction, easy to utilize, and can be readily deployed in a variety of conventional surgical procedures.

### BRIEF SUMMARY OF THE INVENTION

The present invention specifically addresses and alleviates the above-identified deficiencies in the art. Specifically, the present invention is directed to devices and methods for harvesting a segment of tissue from a patient (or donor) that enables a precise segment of tissue to be derived via a quick, simple surgical procedure. According to a preferred embodiment, the device comprises a devise body having an upper and lower elongate housing portions having proximal and distal ends. Preferably the upper and lower portions are formed as mirror images of one another and define a recess or cavity therebetween, the latter of which being operative to retain a segment of tissue harvested from a tissue mass. Formed upon the distal-most end of the device is a cutting edge, the latter of which preferably comprises upper and lower cutting blades formed upon the distal-most ends of the upper and lower housing portions, respectively. In this regard, the upper and lower cutting edges are positioned to be advanced through a tissue mass and shear or slice a layer of tissue therefrom such that the sliced/sheared tissue advances proximally within the recess defined between the upper and lower housing portions. The device may be formed such that the length and width of tissue to be derived will have the desired surface area. Likewise, the cutting edge can be configured such that the thickness of tissue cut thereby is selectively controlled. With respect to such cutting, it is contemplated that such cutting edges may be either formed to be static or stationary, or otherwise include an articulating element to thus enable a segment of tissue sliced or sheared from a tissue mass to be selectively cut from the tissue mass at a selected point.

It is therefore an object of the present invention to provide a tissue harvesting device and method that are operative to quickly, accurately and easily harvest a segment of tissue from a tissue mass such that the harvested tissue possesses a desired surface area and thickness.

Another object of the present invention is to provide a system and method for harvesting a segment of tissue from a patient (or donor) that can be specifically adapted to harvest tissue from any of a variety of target sites about the body.

Another object of the present invention is to provide a device and method for harvesting a segment of tissue from a patient (or donor) that can readily enable a segment of tissue to be harvested that can be immediately utilized in a further surgical procedure.

A still further object of the present invention is to provide a device and method for harvesting a segment of tissue from a patient (or donor) wherein the device is of simple construction, easy to use, and can be manufactured at relatively low cost.

### BRIEF DESCRIPTION OF THE DRAWINGS

These as well as other features of the present invention will become more apparent upon reference to the drawings.

FIG. 1 is a partial perspective view of a leg of a patient illustrating the opposed ends of a segment of tissue being harvested from a pair of incisions formed upon the patient's leg.

FIG. 2 is a perspective view of device formed in accordance with a preferred embodiment of the present invention operative to harvest a segment of tissue through an incision formed upon a patient's leg.

FIG. 3 is a perspective view, shown partially in phantom, of a segment of tissue being harvested from the leg of a patient through an incision formed therein via the usage of the device of the present invention depicted in FIG. 2.

FIG. 4 is a cross-sectional view taken along line 4-4 of FIG. 3.

FIG. 5 is an expanded cross-sectional view of the encircled portion of FIG. 4.

FIG. 6 is the expanded view of FIG. 5 showing a segment of tissue being cut and separated from a tissue mass utilizing the device of the present invention.

FIG. 7 is a cross-sectional view of a segment of tissue being withdrawn from the tissue harvesting device of the present invention.

FIG. 8 is a side view, shown partially in cross-section of a segment of tissue being cut from a tissue mass via an alternative embodiment of the present invention.

### DETAILED DESCRIPTION OF THE INVENTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

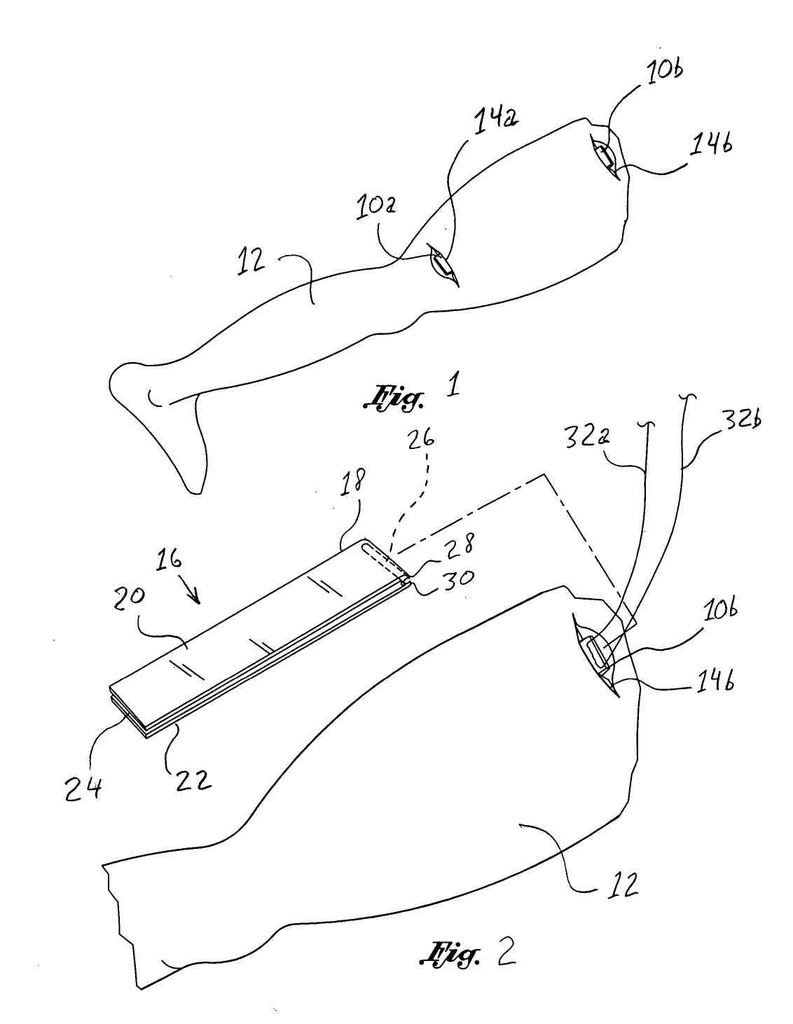

Referring now to the figures, and initially to FIG. 1, there is shown the opposed ends 10a, 10b of a segment of tissue harvested from the leg 12 of a patient through incisions 14a, 14b formed thereupon. As discussed above, a wide variety of surgical procedures employ the use of harvested tissue segments, especially in surgical applications involving the fabrication of slings, grafts for hernia repair or to use as a tissue filler that optimally utilize tissue harvested from a patient or a donor. However, despite the optimal use of tissue for such procedures, there has not heretofore been available any device and/or method by which a segment of tissue can be easily and readily harvested from a patient or donor. In this regard, under conventional practices, in order to derive a desired segment of tissue for use as an implant, sling, graft or in some other context typically requires complex, time-consuming surgical procedures whereby a specific segment of tissue must be precisely excised from the patient or donor. Such procedures are well-known in the art to require exact precision from the surgeon performing such procedure.

To address such shortcomings, there is provided a device 16, illustrated in FIG. 2, that is operative to quickly, easily and accurately harvest a layer of tissue from a patient or donor that can be later utilized in a variety of surgical applications. As illustrated, the device 16 comprises a body portion 18 defined by a top housing portion or section 20 and bottom housing portion or section 22 that are preferably formed as mirror images of one another that define space or cavity 24 therebetween. Formed at the distal most end of the body member 18 is a cutting element 26 defined by cutting elements or blades 28, formed at the distal most end of top portion 20 and lower cutting blade 30 formed at the distal most end of lower portion 22. As illustrated, such cutting element 26 is designed to be inserted into an incision, in this case incision 14b formed upon leg 12, to thus facilitate the harvesting of a segment of tissue, a portion of which is illustrated as 10b extending from incision 14b to which are attached sutures 32a, 32b, the latter for use in the ultimate surgical placement or implantation of such harvested tissue.

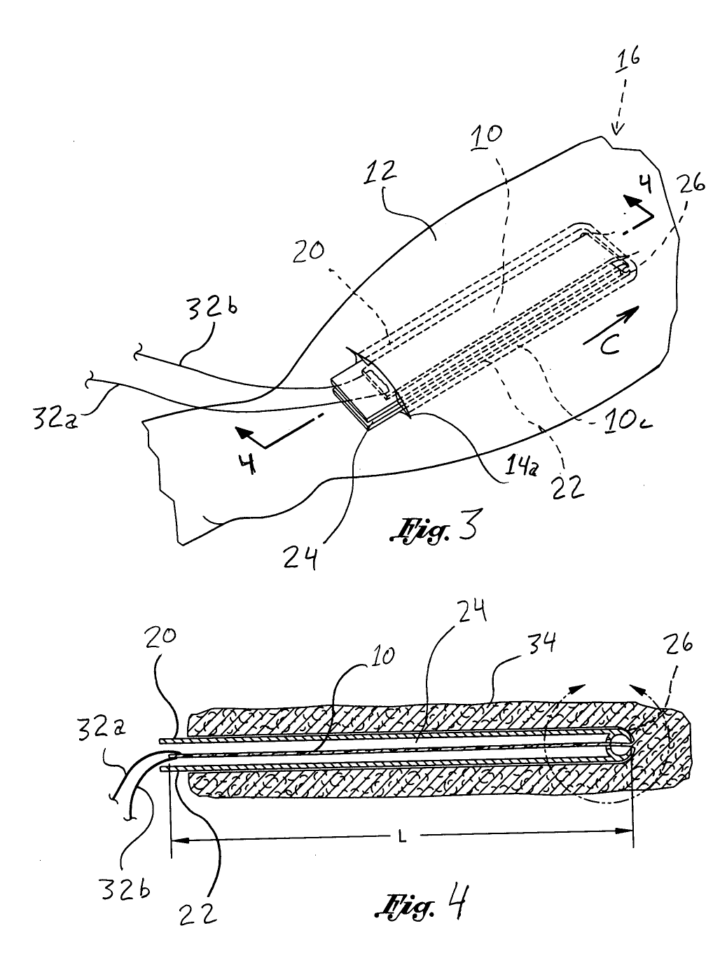

As more clearly illustrated in FIG. 3, device 16 is operative to cut an elongate segment of tissue 10 by merely being inserted through an incision 14a and advanced therethrough such that a segment of tissue 10 becomes cut and disposed within cavity 24 formed between upper and lower portions 20, 22 of the device. In this regard, the forward advancement of the device causes cutting element 26 to slice a section of tissue and thereafter cause the same to be retained within cavity 24 by merely advancing the same in the direction indicated by the letter “C”.

Such cutting motion is more clearly illustrated in FIG. 4 which shows cutting element 26 cutting through a mass of tissue 34 such that a sliced portion 10 thereof is retained within cavity 24. As will be readily appreciated, although shown being advanced through a portion of a patient's leg 12, it will be understood that the same can be deployed anywhere in the body where it may be suitable to derive a particular segment of tissue. Accordingly, it should be understood that the devices of the present invention may be configured in a variety of embodiments operative to harvest a particular portion of tissue and may include housing portions 20, 22 that are either longer, shorter, wider, arcuate or otherwise formed to harvest a particular segment of tissue having an approximate size. Likewise, cutting element 26 will be operative to cut through a desired portion of tissue such that the desired amount and area of tissue is captured for a particular procedure. In the embodiment shown in FIG. 4, upper and lower portions 20, 22 define a length “L” of a graft 10 to which sutures 32a, 32b may be attached and thereafter utilized for a particular procedure. As discussed above, it will be understood by those skilled in the art that the size and shaping of such dimensions can be readily altered to fit a particular harvesting application.

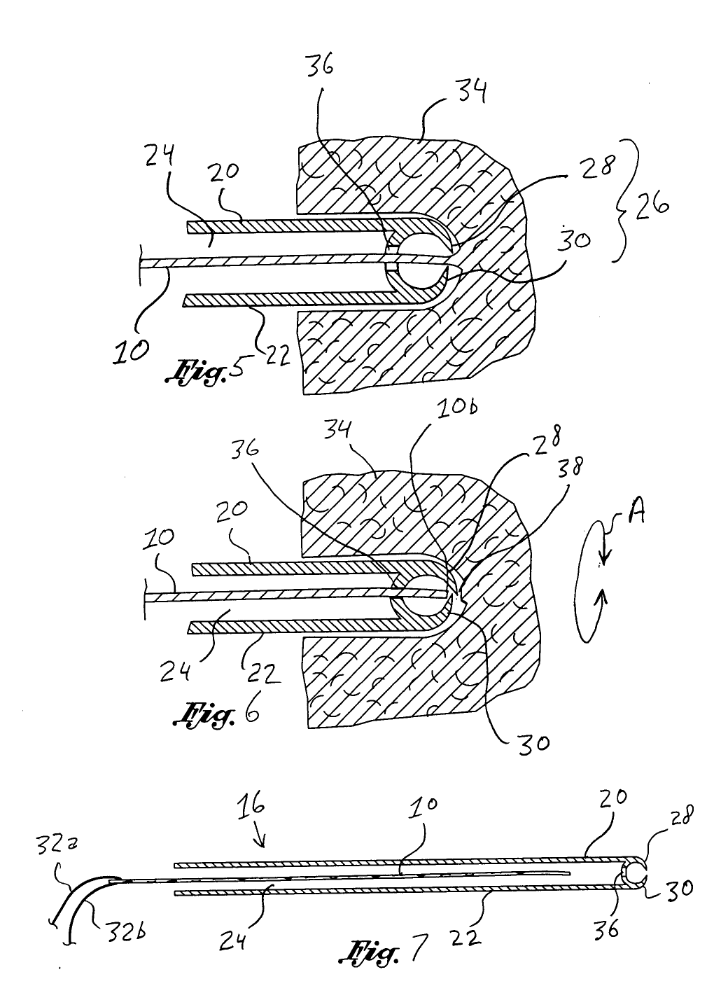

To facilitate such harvesting, cutting element 26 will be specifically adapted to easily and readily cut through and derive a layer of tissue to have the desired thickness and length, as will be readily seen with reference to FIGS. 5 and 6. With respect to the former, cutting blades 28, 30 will be specifically sized to cut through tissue 34 such that a desired thickness of tissue is sheared-off thereby. To effectuate adequate capture, it will be understood that a recess 36 will be provided through which the sheared tissue 10 can thereafter be retained in recess 24 formed between upper and lower portions 20, 22. As will be readily appreciated by those skilled in the art, cutting blades 28, 20 which define cutting element 26 may take any of a variety of configurations and may either be rigidly fixed in position, as shown, or otherwise operative to articulate and cut across a given segment of tissue discussed more fully below. Moreover, it is expressly contemplated that cutting element 26 may only utilize a single blade that is operative to divide the fascia tissue, as a fasciotomy. Along these lines, in such single-blade embodiment (not shown) it is contemplated that the same may be configured per a conventional plane utilized in carpentry that utilizes a single blade operative to divide a fascial segment of tissue to release the underlying muscle compartment. This provides the advantage of avoiding a large external incision on the skin while releasing the fascia effectively.

Once a desired amount of tissue is cut and retained within the device, the cutting blades 28, 30 may be manipulated to thus sever off the segment of tissue 10 so that the same can be withdrawn from tissue mass 34. To achieve that end, it is contemplated that cutting blades 28, 30 may be operative to form or cut 38 such that the tissue segment 10 is severed from tissue mass 34, which may be accomplished by merely rotating the device such that the cutting blades 28, 20 rotate in the manner indicated the letter “A”. As will be appreciated by those skilled in the art, by simple rotational movement, the cutting edges 28, 30 will be operative to form the cut 38 between tissue mass 34 and harvested tissue segment 10 to thus enable the latter to be withdrawn. It is contemplated that such simple rotational movement may also be utilized in those embodiments where only a single blade is utilized to define cutting element 26.

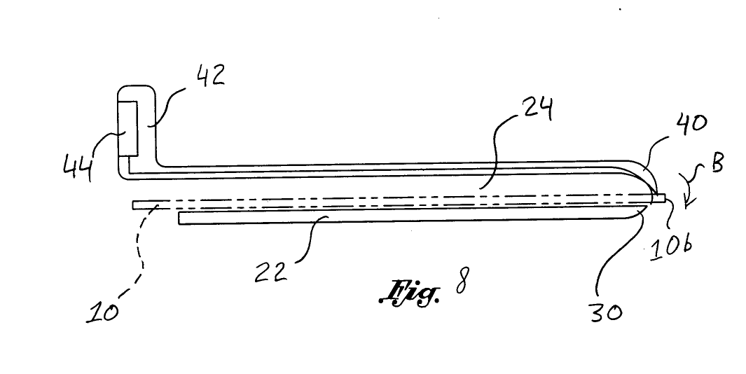

With respect to such withdrawal, there is shown in FIG. 7, the removal of the segment of tissue 10 from the device 16 of the present invention. As illustrated, the segment of tissue 10 to be utilized as a sling, graft or the like, is merely withdrawn from the recess 24 defined between upper and lower segments 20, 22. To facilitate that end, it is contemplated that sutures 32a, 32b may be secured to the proximal-most end of the tissue segment 10 to thus facilitate the reattachment of the same via a later procedure. As will be readily appreciated by those skilled in the art, such segment of tissue 10 may be deployed per any well-known surgical procedure currently practiced or later developed.

Additional modifications and improvements of the present invention may also be apparent to those of ordinary skill in the art. For example, it will be readily understood that the device 16 of the present invention may be sized and adapted to cut through any type of tissue as may be desired for a particular application, as discussed above. Accordingly, however the device 16 of the present invention may be ultimately deployed to advance through a given segment of tissue should be construed as broadly as possible, whether the instrument in moved in a proximal, distal, lateral, or diagonal manner relative a given anatomical structure. Likewise, it is contemplated that the cutting element 26 may be provided with static or rigid cutting blades 28, 30, as illustrated in FIGS. 2-7, or may be provided with an articulating shearing apparatus to facilitate the ability of the device 16 to cut a segment of tissue 10 from the tissue mass from which the same is derived. Such contemplated embodiment is depicted in FIG. 8 which illustrates a segment of tissue 10 drawn into recess 24 formed atop lower portion 22 with a cutting blade 40 formed on the distal-most end of the device operative to impart a downward shearing cut, as illustrated by the letter “B” to the extent a manually operable member defined by handle portions 42, 44 formed on the distal-most end of the device are actuated. As a consequence, a cut is formed at distal-most end 10b of the graft 10 to thus enable the same to be withdrawn and utilized as per the aforementioned procedure. Thus, the particular combination of parts and steps described and illustrated herein is intended to represent only certain embodiments of the present invention, and is not intended to serve as limitations of alternative devices and methods within the spirit and scope of the invention.

1. A device for harvesting a segment of tissue from a tissue mass comprising:
a. a first elongate housing portion having proximal and distal ends;
b. a second elongate housing portion having proximal and distal ends, said second panel member being rigidly affixed to said first elongate housing portion such that said housing portions extend in generally parallel relation to one another and a recess is formed intermediate said first and second panel members; and
c. a cutting element formed upon said distal ends of said first and second panel members, said cutting element being operative to form a sliced segment of tissue when advanced through said tissue mass, said sliced segment of tissue being retained within said recess formed intermediate said first and second panel portions.

2. The device of claim 1 wherein said cutting element is defined by a first blade portion formed upon the distal-most end of said first panel member and a second blade portion formed upon the distal-most end of said second panel member, said first and second cutting edges being cooperatively operative to form a sliced segment of tissue having a fixed thickness.

3. The device of claim 1 wherein said first and second housing portions are formed to have a generally rectangular shape.

4. The device of claim 2 wherein said device further comprises a channel member disposed between said first and second cutting edges, said channel defining a pathway through which said sliced segment of tissue extends proximally within said recess formed intermediate said first and second housing portions.

5. The device of claim 1 wherein said cutting element further has an articulating cutting member operative to slice across and separate a segment of tissue sliced from said tissue mass.

6. A method for harvesting tissue from a patient comprising the steps:
a. providing a device for harvesting tissue from an individual, said device comprising:
(i) a first elongate housing portion having proximal and distal ends;
(ii) a second elongate housing portion having proximal and distal ends, said second panel member being rigidly affixed to said first elongate housing portion such that said housing portions extend in generally parallel relation to one another and a recess is formed intermediate said first and second panel members; and
(iii) a cutting element formed upon said distal ends of said first and second panel members, said cutting element being operative to form a sliced segment of tissue when advanced through said tissue mass, said sliced segment of tissue being retained within said recess formed intermediate said first and second panel portions;
b. forming an incision upon said individual from which said tissue is to be harvested, said incision being operative to receive said cutting apparatus of said device provided in step (a) when said cutting apparatus is introduced therewithin;
c. advancing said cutting element of said device provided in step (a) within said incision formed in step (b) and advancing said device provided in step (a) through said incision such that said cutting element operatively slices a segment of tissue from a tissue mass surrounding said incision such that said sliced segment of tissue is retained within said recess defined between said first and second housing portions;
d. severing said tissue segment sliced from said tissue mass formed in step (c); and
e. retrieving said segment of tissue separated from said tissue mass in step (d).

7. The method of claim 6 further comprising the step;
a. tying at least one suture to said segment of tissue harvested in step (e).

8. The method of claim 6 wherein in step (d), said tissue sliced from said tissue mass is separated from said tissue mass by severing said sliced tissue with said cutting element of said device.

9. The method of claim 8 wherein said cutting element comprises a first blade portion formed upon the distal-most end of said upper housing portion and a second blade member formed upon the distal-most end of said second housing portion, said first and second blade portions cooperatively defining said cutting element.

10. The method of claim 6 wherein said cutting element comprises an articulating blade member operative to sever said sliced segment of tissue cut by said cutting element and retained within said recess formed between said first and second housing portions.

11. The device of claim 1 wherein said cutting element is defined by a single blade formed upon a respective one of said first and second panel members.

12. The method of claim 8 wherein said cutting element comprises a single blade formed upon a respective one of said first and second panel members.
